(19) United States
(12) Patent Application Publication
(10) Pub. No.: US 2024/0366097 A1
(43) Pub. Date: Nov. 7, 2024

(54) HEMODYNAMIC STATE ESTIMATION METHOD

(71) Applicant: Murata Manufacturing Co., Ltd., Nagaokakyo-shi (JP)

(72) Inventor: Toru SHIMUTA, Nagaokakyo-shi (JP)

(21) Appl. No.: 18/773,900

(22) Filed: Jul. 16, 2024

Related U.S. Application Data
(63) Continuation of application No. PCT/JP2023/004840, filed on Feb. 13, 2023.

(30) Foreign Application Priority Data
Feb. 28, 2022 (JP) 2022-030119

(51) Int. Cl. A61B 5/021; A61B 5/00; G16H 50/30

(52) U.S. Cl. CPC A61B 5/02125; A61B 5/4809; A61B 5/4815; A61B 5/6826; A61B 5/684; A61B 5/7235; A61B 5/7275; G16H 50/30; A61B 2560/0462; A61B 2562/0219

(57) ABSTRACT

A system and methods is provided for estimating a peripheral hemodynamic state with high accuracy. An exemplary method includes obtaining a first photoplethysmographic signal of a capillary of a peripheral of a user; obtaining a second photoplethysmographic signal of an arteriole of the peripheral; estimating a pulse wave transmission time based on the first and second photoplethysmographic signals; and estimating a peripheral hemodynamic state of the peripheral based on the pulse wave transmission time. The first and second photoplethysmographic signals are obtained from a predetermined finger of the user.

### CROSS REFERENCE TO RELATED APPLICATIONS

The present application is a continuation of PCT/JP2023/004840 filed Feb. 13, 2023, which claims priority to Japanese Patent Application No. 2022-030119, filed Feb. 28, 2022, the entire contents of each of which are incorporated herein by reference.

### TECHNICAL FIELD

The present disclosure relates to a method for estimating the hemodynamic state of a user.

### BACKGROUND

Currently, the pulse wave transmission time, which is the time for which a pulse wave is transmitted in an artery of the user, can be used as an index for estimating the health state of a user. The pulse wave transmission time is varied in accordance with a change in the blood pressure of a user at a measurement point. International Publication No. WO 2018/030380 (hereinafter “Patent Document 1”) discloses a blood pressure state measurement device for accurately measuring the circulatory dynamic state including the blood pressure state of an arteriole, which is finer than an artery, or that of a capillary in order to estimate the risk of cardiovascular diseases.

For the estimation of the circulatory dynamic state (hemodynamic state) disclosed in Patent Document 1, a photoplethysmographic signal measured from an arteriole or capillaries and a biological signal, which is the basis for the measurement of the pulse wave transmission time, are used. The biological signal, which serves as the measurement basis, is a signal used for estimating the transmission time of a pulse wave from a heart to an artery, which transports blood to arterioles or capillaries. In Patent Document 1, the circulatory dynamic state is estimated based on the pulse wave transmission time.

The blood pressure state measurement device disclosed in Patent Document 1 can be used to estimate the hemodynamic state of a peripheral blood vessel (e.g., the peripheral hemodynamic state). However, with this measurement device, the pulse wave transmission time is greatly varied because of significant differences in the lengths of arterioles and capillaries, which form a transmission path of a pulse wave, among individuals, such as users, and depending on the mounting position of the device. This makes it difficult for this measurement device to estimate the peripheral hemodynamic state from the value of the pulse wave transmission time. As a result, the estimation accuracy of the peripheral hemodynamic state is reduced.

### SUMMARY OF THE INVENTION

In view of the foregoing, it is an object of the present disclosure to provide a method and system for estimating the peripheral hemodynamic state with high accuracy.

In an exemplary aspect, a method is provided that is executed by a biological information measurement system. The exemplary method includes obtaining a first photoplethysmographic signal of a capillary of a peripheral of a user; obtaining a second photoplethysmographic signal of an arteriole of the peripheral; estimating a pulse wave transmission time based on the first and second photoplethysmographic signals; and estimating a peripheral hemodynamic state of the peripheral based on the pulse wave transmission time. In this aspect, the first and second photoplethysmographic signals are obtained from a predetermined finger of the user.

According to the present disclosure, the method and system enable the peripheral hemodynamic state to be estimated with high accuracy.

### BRIEF DESCRIPTION OF DRAWINGS

FIG. 1 is a block diagram illustrating the configuration of a biological information measurement system according to an exemplary embodiment.

FIG. 2 illustrates the external configuration of a sensing device according to the exemplary embodiment.

FIG. 3 illustrates an example of the posture of a user when biological information is measured.

FIG. 4 is a schematic view for explaining the measurement of a photoplethysmographic signal using the sensing device according to the exemplary embodiment.

FIG. 5 is a graph for explaining pulse wave feature values.

FIG. 6 is a graph for explaining the estimation of a pulse wave transmission time based on first and second photoplethysmographic signals.

FIG. 7 is a graph illustrating the correlation between the pulse wave transmission time and the first photoplethysmographic signal.

FIG. 8 is a graph illustrating the correlation between the pulse wave transmission time and the systolic blood pressure.

FIG. 9 is a graph illustrating the correlation between the pulse wave transmission time and a peripheral blood pressure index.

FIG. 10 is a graph illustrating the correlation between the reciprocal of the pulse wave transmission time and the peripheral blood pressure index.

FIG. 11 is a graph illustrating the correlation between the pulse wave transmission time and the peripheral blood pressure index regarding subjects with a disease and those without.

FIG. 12 is a graph illustrating the correlation between the reciprocal of the pulse wave transmission time and the peripheral blood pressure index regarding subjects with a disease and those without.

FIG. 13 is another graph illustrating the correlation between the pulse wave transmission time and the peripheral blood pressure index.

FIG. 14 is another graph illustrating the correlation between the reciprocal of the pulse wave transmission time and the peripheral blood pressure index.

FIG. 15 is another graph illustrating the correlation between the pulse wave transmission time and the peripheral blood pressure index.

FIG. 16 is another graph illustrating the correlation between the reciprocal of the pulse wave transmission time and the peripheral blood pressure index.

FIG. 17 is a flowchart illustrating an example of processing for a peripheral hemodynamic state estimation method according to the exemplary embodiment.

FIG. 18 is a flowchart illustrating another example of processing for the peripheral hemodynamic state estimation method according to the exemplary embodiment.

FIG. 19 is a flowchart illustrating another example of processing for the peripheral hemodynamic state estimation method according to the exemplary embodiment.

### DETAILED DESCRIPTION OF EMBODIMENTS

An exemplary embodiment will be described below with reference to the drawings. The same element is designated by like reference numeral and the same explanation will not be repeated.

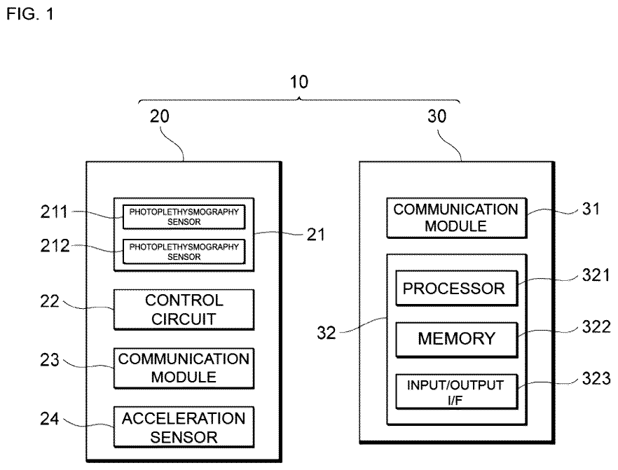

FIG. 1 is a block diagram illustrating the configuration of a biological information measurement system 10 according to the exemplary embodiment. The biological information measurement system 10 includes a sensing device 20 and a computer 30. The sensing device 20 is configured to measure biological information of a user (e.g., a subject). The computer 30 can also be configured to communicate with the sensing device 20.

The sensing device 20 is a wearable device, for example, which can be worn on a peripheral part (a finger, for example) of a user. The sensing device 20 includes a biometric sensor 21, a control circuit 22, a communication module 23, and an acceleration sensor 24. The biometric sensor 21 is configured to measure biological information from a peripheral part (a finger, for example) of a user. The control circuit 22 is configured to control the operation of the biometric sensor 21. The communication module 23 sends measurement results obtained by the sensing device 20 to the computer 30 via a wireless network or a wired network. The acceleration sensor 24 is configured to measure the movement acceleration of the sensing device 20.

The biometric sensor 21 includes photoplethysmography sensors 211 and 212 that measure the index value representing the peripheral blood pressure of a user. According to the exemplary embodiment, the peripheral blood pressure is defined as the blood pressure of peripheral capillaries or arterioles. In general, an arteriole is a fine artery having a diameter of about 20 to 200 μm and is a blood vessel located between an artery and capillaries. Moreover, capillaries are fine blood vessels each having a diameter of about 10 μm and link an artery and a vein with each other.

In an exemplary aspect, a reflection-type photoplethysmography sensor includes a light-emitting element and a light-receiving element. The reflection-type photoplethysmography sensor is configured to apply light having a wavelength range of infrared light or red light or a wavelength range of green light to the body surface of a user from the light-emitting element and measures light reflected by the body surface of the user with the light-receiving element, such as a photodiode or a phototransistor. The blood in an artery contains oxyhemoglobin, which absorbs incident light. The photoplethysmography sensor can thus be configured to measure a photoplethysmographic signal by chronologically sensing the volume of a blood flow which is changed (e.g., volume change in blood vessels) in accordance with the heart pulsation.

The communication module 23 is configured to send measurement results of the sensing device 20 (such as photoplethysmographic signals measured by the photoplethysmography sensors 211 and 212 and the acceleration of the sensing device 20 measured by the acceleration sensor 24) to the computer 30 via a wireless network or a wired network.

The acceleration sensor 24 is configured to measure the movement acceleration of the sensing device 20 when a user changes its posture to measure a pulse wave signal. The acceleration sensor 24 is a three-axis acceleration sensor that detects the direction of the gravitational acceleration. A detection signal obtained by the acceleration sensor 24 can be used for estimating the height of the sensing device 20 worn on the user, the mounting position of the sensing device 20 worn on the user (the position of the user's heart, for example), and the posture of the user, such as a standing posture (standing position), a sitting posture (sitting position), or lying on the back (supine posture).

According to exemplary aspects, the computer 30 can be a multifunction mobile phone, such as a smartphone, or a general-purpose computer (such as a laptop personal computer, a desktop personal computer, a tablet terminal, and a server computer). The computer 30 includes a communication module 31 and a signal processing unit 32. The communication module 31 receives measurement results of the biometric sensor 21 from the sensing device 20 via a wireless network or a wired network. The signal processing unit 32 is configured to estimate biological information of a user from the measurement results of the biometric sensor 21. The signal processing unit 32 includes a processor 321, a memory 322, and an input/output interface 323.

For example, the signal processing unit 32 can be configured to calculate the pulse wave transmission time from photoplethysmographic signals measured by the photoplethysmography sensors 211 and 212 so as to estimate the peripheral hemodynamic state of a user, based on the pulse wave transmission time.

For purposes of this disclosure, the term “pulse wave transmission time” can be used as a time difference between a peak in a pulse wave in an electrocardiogram and that in a measurement point or a time difference between a peak of a pulse wave in a thick artery and that in each measurement point. In the specification, a time difference between a peak of a pulse wave in capillaries in the outer region of the skin and that in an arteriole from which the capillaries branch off will be called the pulse wave transmission time (e.g., a peripheral pulse wave transmission time). Hereinafter, the pulse wave transmission time refers to the peripheral pulse wave transmission time unless otherwise stated.

The signal processing unit 32 can be configured to calculate feature values of a pulse wave from photoplethysmographic signals measured by the photoplethysmography sensors 211 and 212 so as to estimate the peripheral blood pressure index based on the feature values of the pulse wave.

The signal processing unit 32 can also estimate the height of a part of a user wearing the sensing device 20 and the posture of the user, based on a signal from the acceleration sensor 24.

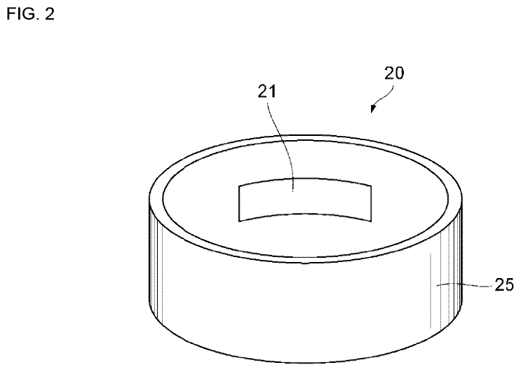

FIG. 2 illustrates the external configuration of the sensing device 20 according to the exemplary embodiment. The sensing device 20 includes a ring-like casing 25 that can be worn by a finger of a user. In the example in FIG. 2, for example, the casing 25 has a hollow tubular shape. The biometric sensor 21 is attached to the inner peripheral surface (e.g., the inner surface of a hollow tube) of the casing 25 so that the ball of a finger of a user faces the biometric sensor 21 when the sensing device 20 is worn on the finger. It is noted that the shape of the casing 25 is not limited to a hollow tubular shape. For example, the casing 25 may have a tubular shape which can fit on a finger of a user (the shape of a finger cone, for example). The tubular casing 25 may have a bottom (a portion contacting a fingertip), though it is not essential. The sensing device 20 may be provided as a portable or stationary electronic device and can be configured to measure a photoplethysmographic signal when a user touches the biometric sensor 21 with a finger.

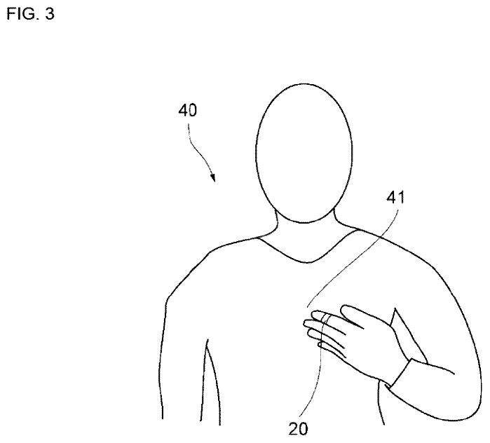

FIG. 3 illustrates an example of the posture of a user 40 when biological information is measured. In this example, the user 40 has brought a finger wearing the sensing device 20 at a standstill at the position of a heart 41, and the sensing device 20 is measuring biological information from the finger of the user 40. It should be appreciated that the position of the sensing device 20 to measure biological information (i.e., the measurement position) is not limited to the heart 41 of the user 40 and may be the face or the abdomen of the user 40. The posture of the user 40 when biological information is measured may be a sitting posture or a supine posture.

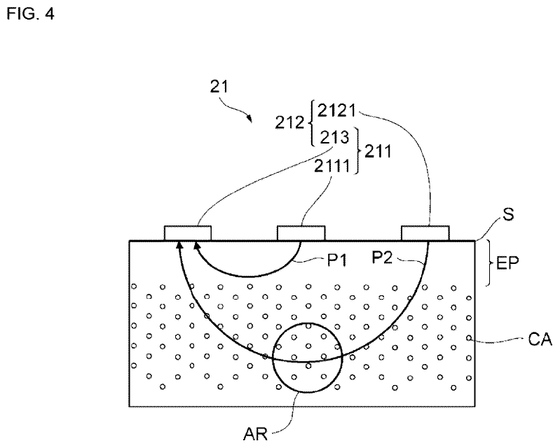

FIG. 4 is a schematic view for explaining the measurement of a photoplethysmographic signal using the biometric sensor 21. FIG. 4 is a schematic sectional view illustrating a state in which the biometric sensor 21 is fixed to the vicinity of the body surface S of a user.

The biometric sensor 21 includes light-emitting elements 2111 and 2121 and a light-receiving element 213. The biometric sensor applies light to the body surface S and receives light absorbed in or reflected by an epidermis region EP, multiple capillaries CA, and an arteriole AR from which the capillaries CA branch off. In the embodiment, one light-receiving element 213 is provided for the light-emitting elements 2111 and 2121. In this case, the light-emitting element 2111 and the light-receiving element 213 form the photoplethysmography sensor 211, while the light-emitting element 2121 and the light-receiving element 213 form the photoplethysmography sensor 212. A light-receiving element may be provided for each of the light-emitting elements 2111 and 2121 in an alternative aspect.

According to the exemplary aspect, the light-emitting element 2111 is an LED or a laser having a wavelength range of about blue to yellow-green light (preferably, wavelengths of 500 to 550 nm). The light-emitting element 2121 is an LED or a laser having a wavelength range of about red to near infrared light (preferably, wavelengths of 750 to 950 nm). The light-emitting element 2111 applies light of a wavelength range that is absorbed into the body intensively, while the light-emitting element 2121 applies light of a wavelength range that is absorbed into the body relatively lightly. The light-receiving element 213 is a photodiode or a phototransistor. A signal generated as a result of light emitted from the light-emitting element 2111 being received by the light-receiving element 213 is a first photoplethysmographic signal. A signal generated as a result of light emitted from the light-emitting element 2121 being received by the light-receiving element 213 is a second photoplethysmographic signal.

As shown, the light-emitting element 2111 is disposed closer to the light-receiving element 213 than the light-emitting element 2121. For example, it is preferable that the distance between the light-emitting element 2111 and the light-receiving element 213 be about 1 to 3 mm, and the distance between the light-emitting element 2121 and the light-receiving element 213 be about 5 to 20 mm. As a result of disposing the light-emitting element 2111 closer to the light-receiving element 213 than the light-emitting element 2121, a light-receiving signal obtained from light emitted from the light-emitting element 2111 indicates more information on the outer region of the skin than that obtained from light emitted from the light-emitting element 2121.

Light emitted from the light-emitting element 2111 is absorbed by the epidermis region EP and the nearby capillaries CA, and transmitted light or reflected light is detected by the light-receiving element 213. Light emitted from the light-emitting element 2121 is absorbed by the epidermis region EP, the capillaries CA, and the arteriole AR, which is located closer to the inside of the body than the epidermis region EP and is detected by the light-receiving element 213. In FIG. 4, light emitted from the light-emitting element 2111 is schematically shown as light along an optical path P1, while light emitted from the light-emitting element 2121 is schematically shown as light along an optical path P2.

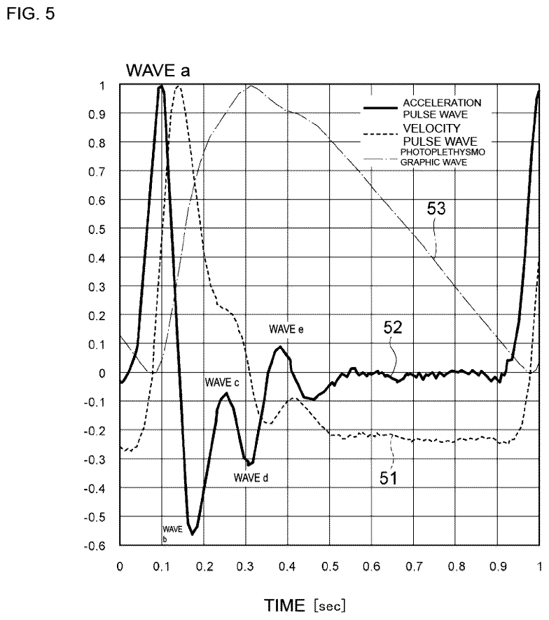

Feature values of a pulse wave will now be explained below with reference to FIG. 5. An explanation will be given below by the use of data obtained by the sensing device 20 of a finger-worn type under the following conditions. An LED having a wavelength of green light (about 525 nm) is used as the light-emitting element 2111. An LED having a wavelength of near infrared light (about 940 nm) is used as the light-emitting element 2121. A silicon photodiode is used as the light-receiving element 213.

Reference numeral 51 denotes a velocity pulse wave signal obtained by performing first-order differentiation on a photoplethysmographic signal. Reference numeral 52 denotes an acceleration pulse wave signal obtained by performing second-order differentiation on a photoplethysmographic signal. The peaks (maximal peaks and minimal peaks) of the acceleration pulse wave signal 52 are called wave a, wave b, wave c, wave d, and wave e, as shown in FIG. 5. Reference numeral 53 denotes a photoplethysmographic signal. Examples of the feature values of a pulse wave that can be used are a peak time difference between peaks (wave a, wave b, wave c, wave d, and wave e), the height of each peak, the ratio of each peak time difference to the pulsation interval, the peak half width, the ratio between the area of the positive side of wave a, wave b, wave c, wave d, and wave e of the acceleration pulse wave signal 52 and that of the negative side thereof, and the degree of matching between the measured pulse waveform and a template pulse waveform. As a feature value of a pulse wave, not only the feature value of every beat, but also the average and/or the standard deviation of the features values of about several beats to dozens of beats may be used.

Among the feature values of a pulse wave, those that are susceptible to the influence of the contact state and the pressing state between the biometric sensor 21 and the skin are feature values regarding the signal strength, such as the height of a pulse wave and the height of each of wave a, wave b, wave c, wave d, and wave e of an acceleration pulse wave. In comparison with such feature values, those which are less susceptible to the influence of the contact state and the pressing state between the biometric sensor 21 and the skin are feature values regarding the time, such as the peak times of wave a, wave b, wave c, wave d, and wave e. Calculating the index representing the level of the peripheral blood flow volume or the level of the peripheral blood pressure of a user from the feature values regarding the time can make the index less likely to be susceptible to the influence of the contact state and the pressing state between the biometric sensor 21 and the skin. For purposes of this disclosure, the index representing the level of the peripheral blood flow volume or the level of the peripheral blood pressure will be called the peripheral hemodynamic state index. Hereinafter, the blood flow volume refers to the peripheral blood flow volume unless otherwise stated.

Typically, the peripheral blood pressure is lowered from the systolic blood pressure measured on a wrist due to the vascular resistance between the wrist and a peripheral blood vessel. The vascular resistance between the wrist and a peripheral blood vessel can be regarded as being substantially constant if the height of the measurement point from the heart is merely adjusted. The peripheral blood pressure is thus proportional to the systolic blood pressure on the wrist. It is thus likely that the peripheral blood pressure index is substantially proportional to the systolic blood pressure if the height of the measurement point from the heart is minimally adjusted.

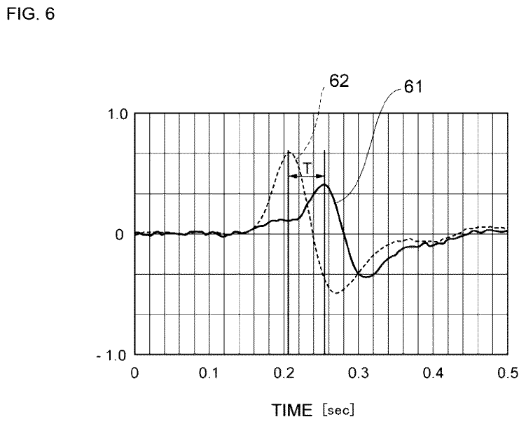

FIG. 6 illustrates an acceleration pulse wave signal 61 generated based on the first photoplethysmographic signal and an acceleration pulse wave signal 62 generated based on the second photoplethysmographic signal. The difference between the time point at the peak of the wave a of the acceleration pulse wave signal 61 and that of the acceleration pulse signal 62 is the pulse wave transmission time T. The reason why such a time point difference is generated is as follows. In general, a pulse wave transmitted from the heart first passes through an artery and reaches an arteriole and then branches off to capillaries. A time difference is thus generated between the time at which the pulse wave reaches the arteriole and the time at which the pulse wave reaches an individual capillary. As a result, a difference between the time point at the peak of the acceleration pulse wave signal 61 and that of the acceleration pulse signal 62 is generated.

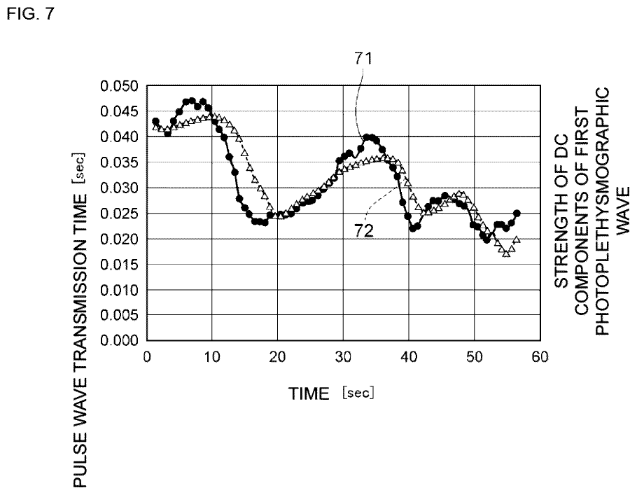

FIG. 7 is a graph illustrating a comparison result between the pulse wave transmission time and DC components of the first photoplethysmographic wave in FIG. 6. A curve 71 representing the transition of the pulse wave transmission time and a curve 72 representing the transition of the DC components of the first photoplethysmographic wave show that the pulse wave transmission time and the DC components of the first photoplethysmographic wave have a correlation. A high strength of the DC components of the first photoplethysmographic wave means a low absorption of light by the blood. In this example, a decreased peripheral blood volume due to a temporary drop in cardiac output is reflected by an increase in the DC components of the first photoplethysmographic wave. When the DC components of the first photoplethysmographic wave, which represent the peripheral blood volume, increase, the pulse wave transmission time having a correlation with the DC components of the first photoplethysmographic wave also increases. Hence, the increased pulse wave transmission time indicates the decreased peripheral blood volume, that is, poor blood circulation. The DC components of the first photoplethysmographic wave are varied depending on the contact state and the pressing state between the sensing device 20 and the skin, resulting in the variations of the DC components among individual measurements. Hence, the direct use of the DC components of the first photoplethysmographic wave to estimate the peripheral hemodynamic state is difficult. Meanwhile, the pulse wave transmission time that is less likely to vary can be used for estimating the peripheral hemodynamic state.

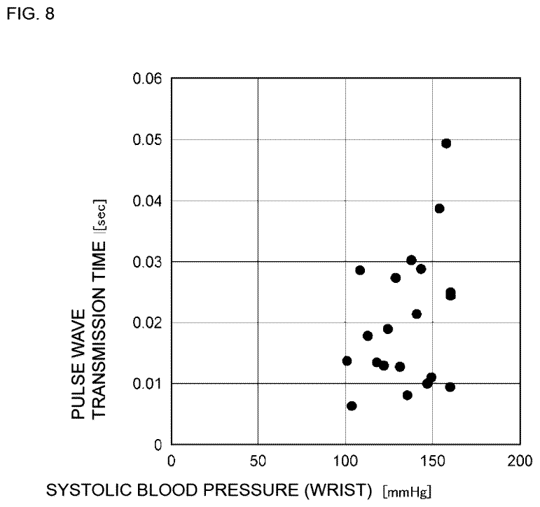

FIG. 8 is a plot graph of the pulse wave transmission time of subjects with respect to the systolic blood pressure of the subjects measured by the biological information measurement system 10. Each point in the graph corresponds to data of one subject. The number of pieces of data is twenty-one in this exemplary aspect.

In an exemplary aspect, the biological information measurement system 10 measures first and second photoplethysmographic signals for thirty seconds in a state in which a subject (e.g., user) is holding the sensing device 20 at the height of the chest (heart). The biological information measurement system 10 then calculates the pulse wave transmission time at each measurement time point, based on the first and second photoplethysmographic signals at the corresponding measurement time point. The biological information measurement system 10 calculates the average of the pulse wave transmission times of a subject at the individual measurement time points as the pulse wave transmission time of the subject. The systolic blood pressure is the blood pressure measured by a cuff-type blood pressure monitor worn on the wrist of a subject. As shown in FIG. 8, no clear correlation is found between the pulse wave transmission time and the systolic blood pressure. That is, it is difficult to estimate the peripheral hemodynamic state, which can be estimated from the pulse wave transmission time, from the measurement of the blood pressure on the wrist.

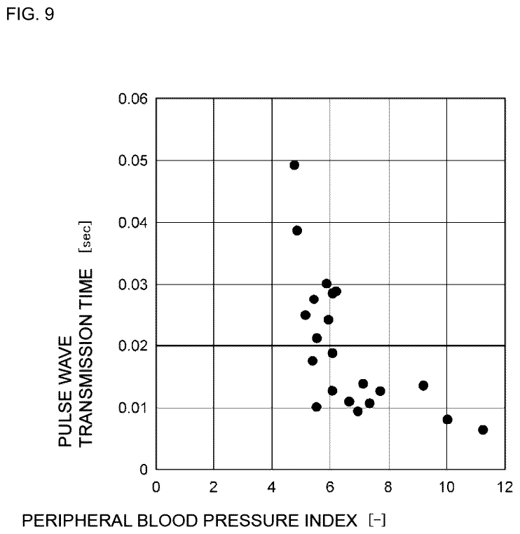

As illustrated in FIG. 9, the peripheral hemodynamic state of a user can be estimated by comparing the pulse wave transmission time and the peripheral blood pressure index, which is the index value representing the level of the peripheral blood pressure of a user, calculated from the feature values of a peripheral pulse wave. FIG. 9 is a plot graph of the pulse wave transmission time with respect to the peripheral blood pressure index when a subject holds the sensing device 20 at the height of the chest. The value of the peripheral blood pressure index is changed in accordance with the peripheral blood pressure.

FIG. 9 shows that, when the peripheral blood pressure index is about seven (i.e., the index value) or smaller, the pulse wave transmission time soars. This suggests that the pulse wave transmission time is increased as a result of the pulse wave transmission velocity being lowered due to a decrease in the peripheral blood pressure index, that is, a drop in the peripheral blood pressure.

In the biological information measurement system 10, the threshold is set for the pulse wave transmission time based on a change in the pulse wave transmission time with respect to the peripheral blood pressure index, and the peripheral hemodynamic state of a user can be estimated based on the threshold. For example, in a range in which the peripheral blood pressure index is larger than about seven, the pulse wave transmission time is about 0.02 sec or shorter. The threshold for the pulse wave transmission time can thus be set to 0.02 sec. In the biological information measurement system 10, if the pulse wave transmission time which exceeds this threshold is measured for a user, it can be estimated that the peripheral hemodynamic state of this user is poor. In the example in FIG. 9, for nine subjects out of twenty-one, the peripheral hemodynamic state is estimated to be poor.

In general, it is possible to determine the peripheral hemodynamic state of a user only based on the pulse wave transmission time. However, the pulse wave transmission time is changed in accordance with the length of a path through which a pulse wave is transmitted. The length of a path is varied depending on the mounting position of a user worn on the sensing device 20 or due to the difference in the biological characteristics among users, such as the sectional area of a finger.

To eliminate the variations in the pulse wave transmission time due to the length of a path, it is desirable to use the pulse wave transmission velocity obtained by dividing the length of the path by the pulse wave transmission time. It is however very difficult to measure the length of an arteriole or capillaries through which a pulse wave is transmitted (i.e., the pulse wave transmission path). Hence, by setting a condition using the peripheral blood pressure index in addition to the pulse wave transmission time, the peripheral hemodynamic state can be estimated with high accuracy while reducing the influence of the above-described variations in the pulse wave transmission time.

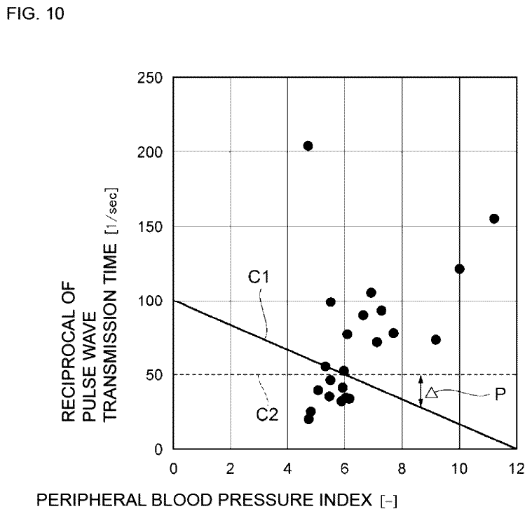

FIG. 10 is a plot graph of the reciprocal of the pulse wave transmission time with respect to the peripheral blood pressure index. As shown in FIG. 10, data points of the subjects are distributed to be able to approximate to a linear expression on a plot plane in FIG. 10.

In one example, a region in the graph of FIG. 10 is divided by a linear conditional expression that connects a point (12, 0) at which the peripheral blood pressure index is 12 and the reciprocal of the pulse wave transmission time is 0 and a point (0, 100) at which the peripheral blood pressure index is 0 and the reciprocal of the pulse wave transmission time is 100. Then, a condition using the pulse wave transmission time and the peripheral blood pressure index can be determined based on this region. That is, it can be estimated whether the peripheral hemodynamic state is good or poor in accordance with the position of a point on a plot plane, which is determined by the peripheral blood pressure index and the reciprocal of the pulse wave transmission time of a user, in the region divided by the conditional expression. For example, in FIG. 10, based on a straight line C1 representing the conditional expression used as a boundary, if a point on the plot plane is on the side of the origin, the peripheral hemodynamic state is estimated to be poor. In the other cases, the peripheral hemodynamic state is estimated to be good. In the example in FIG. 10, for nine subjects out of twenty-one, the peripheral hemodynamic state is estimated to be poor.

The advantage of using the peripheral blood pressure index as a condition for estimating the peripheral hemodynamic state will be explained below. For example, for a virtual data point P in FIG. 10, if the threshold (straight line C2) which is set to 0.02 sec is used for the pulse wave transmission time, as shown in FIG. 9, the peripheral hemodynamic state is estimated to be poor since the data point P is larger than 0.02 sec (the reciprocal is smaller than 50).

As stated above, however, the pulse wave transmission time varies among users. Even if the pulse wave transmission time is large as in the data point P, it may be due to the incorrect mounting position of the sensing device 20 worn by the user, and the actual peripheral blood pressure index may be high and the peripheral hemodynamic state of the user may be estimated to be good. When the peripheral hemodynamic state is estimated with the condition based on the pulse wave transmission time and the peripheral blood pressure index, the peripheral hemodynamic state of the user corresponding to the data point P is estimated to be good.

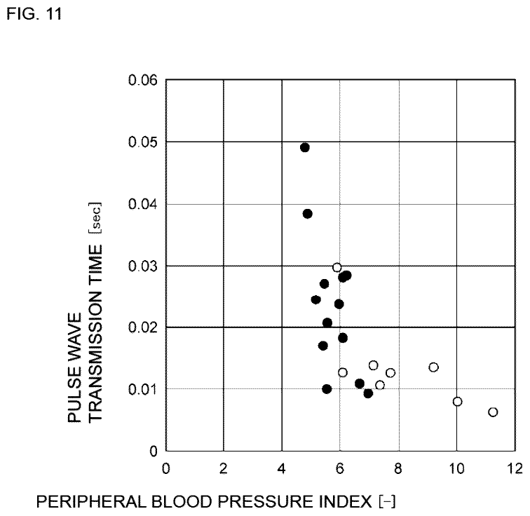

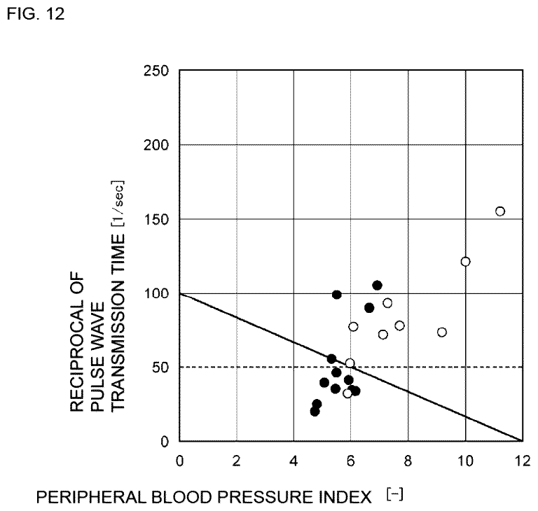

An explanation will be given, with reference to FIGS. 11 and 12, to validate that the above-described estimation method for the peripheral hemodynamic state is proper. FIG. 11 is a graph of the same data as that in FIG. 9 obtained by plotting diabetic patients with the black circles and healthy subjects with the white circles. FIG. 12 is a graph of the same data as that in FIG. 10 obtained by plotting diabetic patients with the black circles and healthy subjects with the white circles.

Diabetes is characterized by sustained high blood sugar levels due to the degradation of carbohydrate metabolism. This causes damage to blood vessels and lowers the vascular endothelial function, which often leads to the hardening of the arteries and the progression of peripheral vascular disorder. The peripheral vascular disorder includes the worsening of the peripheral hemodynamic state. In both of FIGS. 11 and 12, data items from which the peripheral hemodynamic state is estimated to be poor based on the threshold or the condition are mostly those of diabetic patients. That is, the estimation of the peripheral hemodynamic state based on the threshold of the pulse wave transmission time in FIG. 11 and that based on the condition using the pulse wave transmission time and the peripheral blood pressure index in FIG. 12 successfully spot corresponding patients, and it is validated that the above-described estimation method is proper.

Variations in the estimation results of the peripheral hemodynamic state according to the height position of the sensing device 20 with respect to a user will be discussed below.

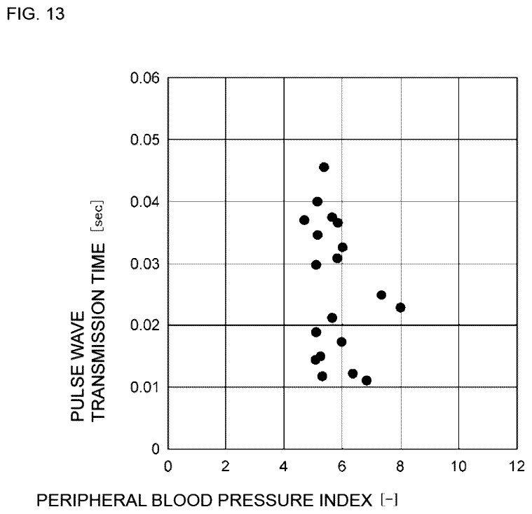

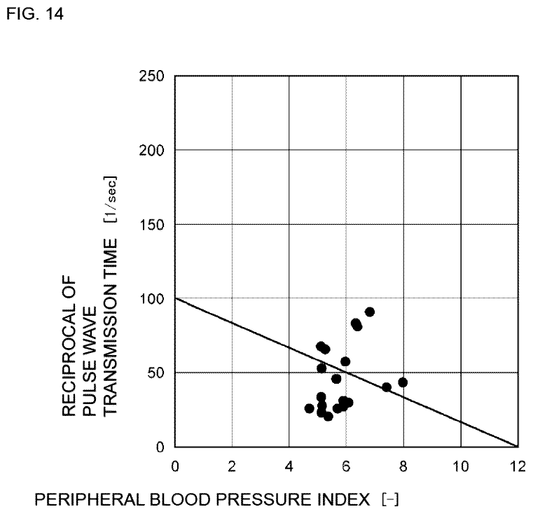

FIG. 13 is a plot graph of the pulse wave transmission time and the peripheral blood pressure index when a subject is in a sitting posture and brings the sensing device 20 close to the head of the subject. FIG. 14 is a plot graph of the reciprocal of the pulse wave transmission time and the peripheral blood pressure index when the subject is in a sitting posture and brings the sensing device 20 close to the head of the subject.

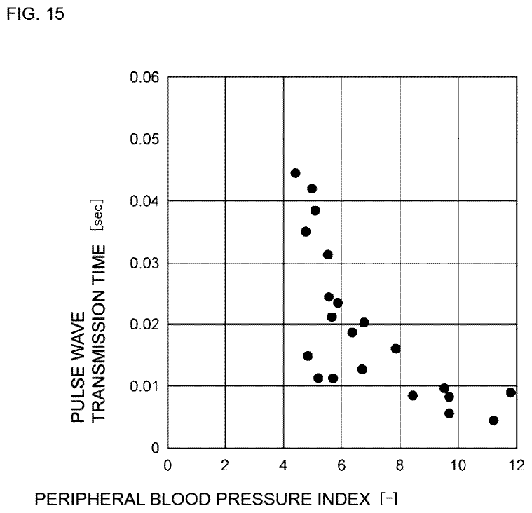

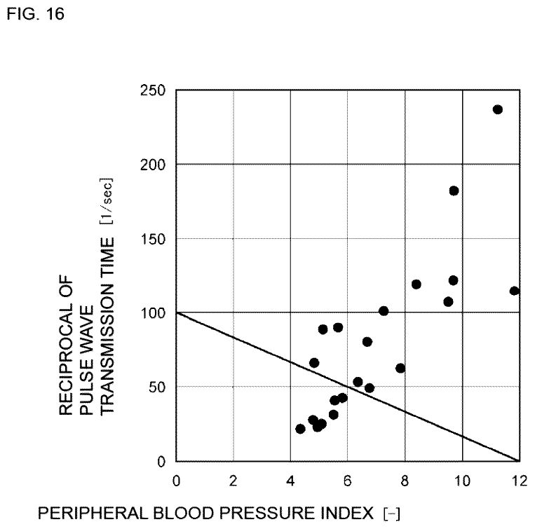

FIG. 15 is a plot graph of the pulse wave transmission time and the peripheral blood pressure index when a subject is in a sitting posture and brings the sensing device 20 close to the abdomen of the subject. FIG. 16 is a plot graph of the reciprocal of the pulse wave transmission time and the peripheral blood pressure index when the subject is in a sitting posture and brings the sensing device 20 close to the abdomen of the subject.

Upon comparing FIGS. 9, 13, and 15 with each other or FIGS. 10, 14, and 16 with each other, it can be seen that, as the relative height of the sensing device 20 to the heart of the subject becomes higher, the number of data items indicating high values of the peripheral blood pressure index becomes smaller and the number of data items indicating large values of the pulse wave transmission time becomes greater. This shows that, as the position of a finger, which is a measurement part wearing the sensing device 20, rises to a higher level, the peripheral blood pressure is lowered due to the pressure produced by the difference in the height with respect to the heart and the pulse wave transmission time is increased. In contrast, regardless of the height of the sensing device 20, the value of the peripheral blood pressure index when the pulse wave transmission time starts to soar is about seven and is almost unchanged. That is, variations in the threshold of the pulse wave transmission time used for estimating the peripheral hemodynamic state are small.

A photoplethysmographic signal is measured by changing the posture of a user to vary the relative height of the sensing device 20 to the heart. This makes it possible to improve the estimation accuracy of the peripheral hemodynamic state. For example, (a) the pulse wave transmission time (e.g., the first pulse wave transmission time) and the peripheral hemodynamic state (e.g., the first peripheral hemodynamic state) are estimated based on a photoplethysmographic signal measured when the sensing device 20 is at the height of the chest; (b) the pulse wave transmission time (e.g., the second pulse wave transmission time) and the peripheral hemodynamic state (e.g., the second peripheral hemodynamic state) are estimated based on a photoplethysmographic signal measured when the sensing device 20 is at the height of the head; and (c) the final peripheral hemodynamic state is estimated based on the first peripheral hemodynamic state and the second peripheral hemodynamic state.

By estimating the peripheral hemodynamic state in this manner, the peripheral hemodynamic state can be estimated in a more stepwise manner. For example, the following three cases are possible: a first case in which the peripheral hemodynamic state is estimated to be good in both of the estimation steps (a) and (b); a second case in which the peripheral hemodynamic state is estimated to be poor in one of the estimation steps (a) and (b); and a third case in which the peripheral hemodynamic state is estimated to be poor in both of the estimation steps (a) and (b). The final peripheral hemodynamic state in the estimation step (c) is estimated in the following manner: the peripheral hemodynamic state is estimated to be good in the first case; the peripheral hemodynamic state is estimated to be relatively poor in the second case; and the peripheral hemodynamic state is estimated to be poor in the third case.

In the above-described example, the peripheral hemodynamic state is estimated by using the results obtained by measuring photoplethysmographic signals at two positions, that is, at the chest and the head. Alternatively, the peripheral hemodynamic state may be estimated by using the results obtained by measuring photoplethysmographic signals at three positions, for example, at the chest, the head, and the abdomen. The combination of the two measurement positions may be the chest and the abdomen or the head and the abdomen.

To change the posture of a user to vary the relative height of the sensing device 20 to the heart, the user may lie on the back on a flat surface and place a hand on the chest and the user may lie on the back on a flat surface and place a hand on the flat surface. When the user places a hand on the chest, the position of the sensing device 20 worn on the hand is higher than the position of the heart. When the user places a hand on the flat surface, the position of the sensing device 20 worn on the hand is lower than the position of the heart. The relative height of the sensing device 20 to the heart can be varied in this manner.

It should be appreciated that the above-described postures are those that the user can easily take, and that the user can repeat to make them highly reproducible. Additionally, by limiting the “abdomen” to the “navel” or the head to the “forehead”, for example, users can repeat the postures to make the reproducibility even higher. This reduces the measurement variations among users.

As discussed above, the pulse wave transmission time and the peripheral blood pressure index are varied in accordance with the relative height of the sensing device 20 to the heart. In the biological information measurement system 10, the computer 30 can be configured to determine the height of the sensing device 20 from the heart and to estimate the peripheral hemodynamic state when the sensing device 20 is at the height of the heart. This limits the relative height of the sensing device 20 to the heart to a certain range. Hence, the peripheral hemodynamic state can be estimated while reducing the influence of changes in the pulse wave transmission time and in the peripheral blood pressure index caused by the difference of the relative height of the sensing device 20 to the heart.

In the biological information measurement system 10, the computer 30 can be configured to estimate the amount of change in the height of the sensing device 20, based on information from the acceleration sensor 24 of the sensing device 20. The computer 30 can then estimate the peripheral hemodynamic state based on the pulse wave transmission time and the amount of change in the height of the sensing device 20. Based on the amount of change, the computer 30 can correct the influence of the variations of the height of the sensing device 20 on the pulse wave transmission time. This configuration improves the estimation accuracy of the peripheral hemodynamic state. The computer 30 may estimate the peripheral hemodynamic state, based on the pulse wave transmission time, the peripheral blood pressure index, and the amount of change in the height of the sensing device 20.

In the biological information measurement system 10, the sensing device 20 can be configured to may continuously or intermittently measure a photoplethysmographic signal from a user sleeping while wearing the sensing device 20. Then, the computer 30 may estimate the pulse wave transmission time or both of the pulse wave transmission time and the peripheral blood pressure index and determine the peripheral hemodynamic state and the quality of sleep from temporal variations (such as the maximum value, frequency of increase, rate of change, amount of change, variance, coefficient of variation, and time at which the maximum value or the minimum value is taken) in the value of the estimated pulse wave transmission time or the values of the estimated pulse wave transmission time and peripheral blood pressure index.

Typically, during sleep, the peripheral blood flow increases, and the peripheral pulse wave transmission time becomes shorter, and the peripheral blood pressure is raised. In spite of this tendency, if, during sleep, the pulse wave transmission time becomes longer or the peripheral blood pressure is lowered, it can be estimated that the peripheral hemodynamic state is poor, and the quality of sleep is low.

During sleep, the peripheral hemodynamic state temporarily varies when a user changes the position of the hand or changes the posture while rolling over, for example, to another posture (such as lying on the back, lying on the lateral side, and lying on the stomach). However, such a temporal change can be ignored, and the need of continuous measurement is not great. Moreover, continuous measurement increases power consumption, and intermittent measurement is more preferable than continuous measurement since a smaller battery in a wearable device is desirable.

Measurement is desirably performed for about five to sixty seconds at the interval of one to ten minutes, for example. This methodology can detect the influence of the waking-up during sleep and/or variations due to the difference between REM sleep and non-REM sleep, which are said to be repeated at a cycle of about 90 minutes for a good quality sleep.

The pulse wave transmission time varies when a measurement part is moving significantly or when a user is taking exercise. In the biological information measurement system 10, therefore, the computer 30 can be configured to determine whether a user is in a resting state based on information obtained by the acceleration sensor 24 of the sensing device 20, and the sensing device 20 can be configured to estimate the peripheral hemodynamic state by using the pulse wave transmission time obtained when the user is in a resting state. Accordingly, the peripheral hemodynamic state can be estimated by reducing the influence of variations in the pulse wave transmission time, which are caused by the difference between when the user is in the resting state and when the user is not in the resting state, thereby improving the estimation accuracy.

In the biological information measurement system 10, the computer 30 determines the sleeping state of a user based on information obtained by the acceleration sensor 24 of the sensing device 20. This can identify when the user falls asleep or awakes while sleeping. When there is a change in the pulse wave transmission time or the peripheral blood pressure index, the computer 30 compares the timing at which such a change occurs and the timing at which the user falls asleep or awakes while sleeping. With this arrangement, even if the pulse wave transmission time or the peripheral blood pressure index is changed when the user wakes up during sleep, for example, such a change does not influence the estimation of the quality of sleep. This improves the estimation accuracy of the quality of sleep.

In the biological information measurement system 10, the sensing device 20 can be configured to continuously or intermittently measure a photoplethysmographic signal over a period of one day or longer. Then, the computer 30 can be configured to estimate the pulse wave transmission time or both of the pulse wave transmission time and the peripheral blood pressure index and determine the peripheral hemodynamic state and the state of the peripheral blood circulation disorder (e.g., the degree or signs of disorder) from variations (such as the maximum value, frequency of increase, rate of change, amount of change, variance, coefficient of variation, and time at which the maximum value or the minimum value is taken) in the value of the estimated pulse wave transmission time or the values of the estimated pulse wave transmission time and peripheral blood pressure index.

Continuation of the poor peripheral hemodynamic state over a long period may cause the peripheral blood circulation disorder. The biological information measurement system 10 thus measures a photoplethysmographic signal of a user over a long period and then estimates the peripheral hemodynamic state of the user, thereby enabling the detection of signs of the peripheral blood circulation disorder.

In this case, as in the measurement of a photoplethysmographic signal while a user is sleeping, the need of continuous measurement is not great and intermittent measurement is preferable. Measurement is desirably performed for about five to sixty seconds at the interval of one to ten minutes, for example. This can detect the influence of variations in the peripheral hemodynamic state caused by events, such as taking exercise or having meals, which may influence the peripheral hemodynamic state.

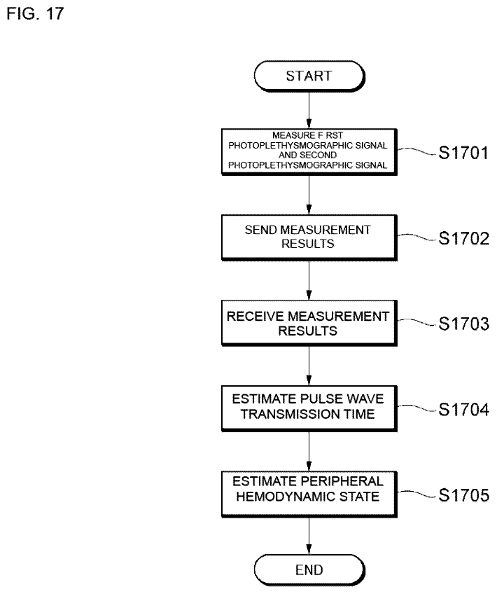

FIG. 17 is a flowchart illustrating an example of processing for a hemodynamic state estimation method according to the exemplary embodiment. Processing of the biological information measurement system 10 is executed, for example, as a result of the sensing device 20 and the computer 30 each including an information processing device, such as a processor, executing a program stored in a non-transitory storage region of the corresponding one of the sensing device 20 and the computer 30.

In step S1701, the sensing device 20 of the biological information measurement system 10 measures a photoplethysmographic signal from a finger of a user wearing the sensing device 20. More specifically, the photoplethysmography sensor 211 measures a first photoplethysmographic signal, while the photoplethysmography sensor 212 measures a second photoplethysmographic signal.

In step S1702, the sensing device 20 sends the measurement results to the computer 30 of the biological information measurement system 10.

In step S1703, the computer 30 receives the measurement results obtained by the sensing device 20.

In step S1704, the computer 30 estimates the pulse wave transmission time of the user. For example, the computer 30 calculates pulse wave feature values from the photoplethysmographic signals measured by the biometric sensor 21 and calculates the pulse wave transmission time of the user from the calculated feature values.

In step S1705, the computer 30 estimates the peripheral hemodynamic state of the user based on the pulse wave transmission time. For example, the computer 30 estimates the peripheral hemodynamic state based on the threshold for the pulse wave transmission time stored in a storage device, such as the memory 322.

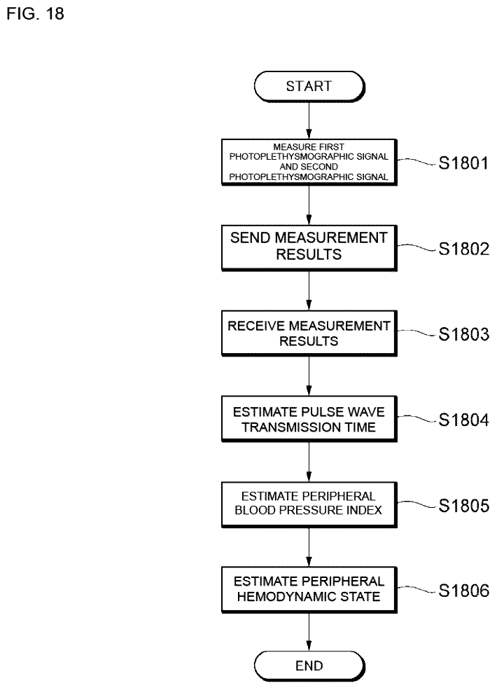

FIG. 18 is a flowchart illustrating another example of processing for the hemodynamic state estimation method according to the exemplary embodiment.

Steps S1801 through S1804 are the same as steps S1701 through S1704.

In step S1805, the computer 30 estimates the peripheral blood pressure index of the user. For example, the computer 30 calculates pulse wave feature values from the photoplethysmographic signals measured by the biometric sensor 21 and calculates the peripheral blood pressure index of the user from the calculated feature values.

In step S1806, the computer 30 estimates the peripheral hemodynamic state of the user based on the pulse wave transmission time and the peripheral blood pressure index. For example, the computer 30 estimates the peripheral hemodynamic state based on the condition using the pulse wave transmission time and the peripheral blood pressure index stored in a storage device, such as the memory 322.

The biological information measurement system 10 may also determine whether the user is sleeping and repeat steps S1701 through S1704 or steps S1801 through S1805 over a period of time while the user is sleeping. The biological information measurement system 10 may continuously or intermittently measure the first and second photoplethysmographic signals of the user multiple times and estimate the pulse wave transmission time or the peripheral blood pressure index multiple times. In step S1705 or S1806, the biological information measurement system 10 may estimate the peripheral hemodynamic state and also the quality of sleep, based on a temporal change in the pulse wave transmission time or peripheral blood pressure index measured multiple times.

The biological information measurement system 10 may repeat steps S1701 through S1704 or steps S1801 through S1805 over the active time period of the user during one day and continuously or intermittently measure the first and second photoplethysmographic signals of the user and estimate the pulse wave transmission time or the peripheral blood pressure index multiple times. In step S1705 or S1806, the biological information measurement system 10 may estimate the peripheral hemodynamic state and also the peripheral blood circulation disorder state, based on a temporal change in the pulse wave transmission time or peripheral blood pressure index measured multiple times.

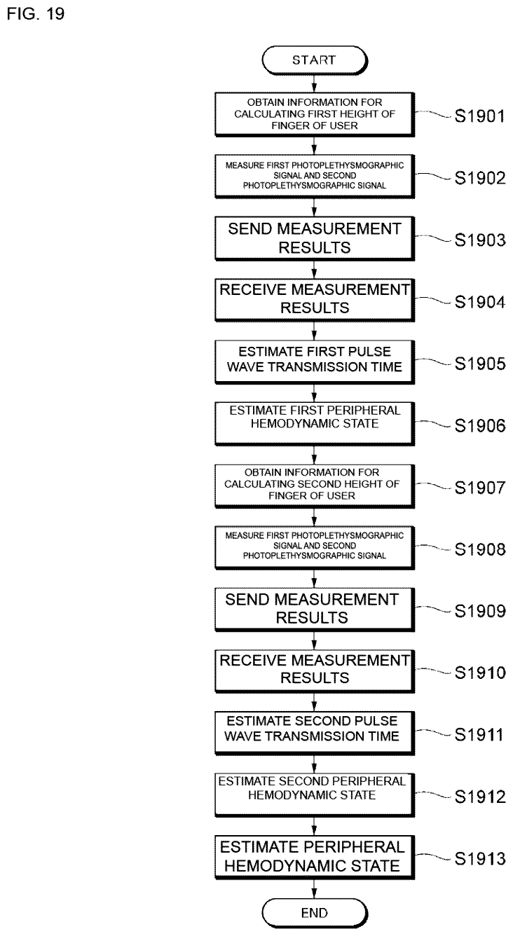

FIG. 19 is a flowchart illustrating another example of processing for the hemodynamic state estimation method according to the exemplary embodiment.

In step S1901, the sensing device 20 of the biological information measurement system 10 obtains information for calculating a first height of a finger of a user wearing the sensing device 20. For example, the sensing device 20 sets the relative height of the finger of the user to the heart to the first height and obtains information for calculating the first height from the acceleration sensor 24.

In step S1902, the sensing device 20 measures a photoplethysmographic signal from the finger of the user wearing the sensing device 20. More specifically, the photoplethysmography sensor 211 measures the first photoplethysmographic signal, while the photoplethysmography sensor 212 measures the second photoplethysmographic signal.

In step S1903, the sensing device 20 sends information on the first height and the first and second photoplethysmographic signals as the measurement results to the computer 30 of the biological information measurement system 10.

In step S1904, the computer 30 receives the measurement results obtained by the sensing device 20.

In step S1905, the computer 30 calculates the first height by using the information for calculating the first height and estimates the pulse wave transmission time of the user corresponding to the first height. In step S1905, the computer 30 estimates the pulse wave transmission time corresponding to the first height as the first pulse wave transmission time.

In step S1906, based on the first pulse wave transmission time, the computer 30 estimates the peripheral hemodynamic state corresponding to the first height as the first peripheral hemodynamic state. The estimation result is temporarily stored in the memory 322, for example.

In step S1907, the sensing device 20 obtains information for calculating a second height of the finger of the user wearing the sensing device 20. For example, the sensing device 20 sets the relative height of the finger of the user to the heart, which is different from the first height, to the second height and obtains information for calculating the second height from the acceleration sensor 24.

Steps S1908 through S1910 are the same as steps S1902 through S1904.

In step S1911, the computer 30 calculates the second height by using the information for calculating the second height and estimates the pulse wave transmission time of the user corresponding to the second height. In step S1911, the computer 30 estimates the pulse wave transmission time corresponding to the second height as the second pulse wave transmission time.

It is noted that obtaining information for calculating the height in steps S1901 and S1907 can be omitted in an exemplary aspect. For example, the computer 30 may obtain information indicating the first height and the second height of the sensing device 20 from the user, for example, and relate the information to the pulse wave transmission time and the peripheral hemodynamic state.

In step S1912, based on the second pulse wave transmission time, the computer 30 estimates the peripheral hemodynamic state corresponding to the second height as the second peripheral hemodynamic state.

In step S1913, the computer 30 estimates the peripheral hemodynamic state based on the first and second peripheral hemodynamic states.

In general, the exemplary embodiment of the invention has been discussed above. The method executed by the biological information measurement system described in the exemplary embodiment is a method that includes obtaining a first photoplethysmographic signal of a capillary of a peripheral of a user; obtaining a second photoplethysmographic signal of an arteriole of the peripheral; estimating a pulse wave transmission time based on the first and second photoplethysmographic signals; and estimating a peripheral hemodynamic state of the peripheral based on the pulse wave transmission time. The first and second photoplethysmographic signals are obtained from a predetermined finger of the user.

As described above, the pulse wave transmission time is estimated based on multiple photoplethysmographic signals obtained from a predetermined finger of a user. As a result, the pulse wave transmission time can be estimated by reducing variations of the estimation results of the pulse wave transmission time, which are caused by different lengths of capillaries and arterioles, which form a pulse wave transmission path, among individuals, such as users, and depending on the mounting position of the device. It is thus possible to accurately estimate the pulse wave transmission time and to estimate the peripheral hemodynamic state based on the pulse wave transmission time, thereby improving the estimation accuracy of the peripheral hemodynamic state.

The above-described method may further include estimating a peripheral blood pressure index of the user. The estimating of the peripheral hemodynamic state may be estimating of the peripheral hemodynamic state based on the pulse wave transmission time and the peripheral blood pressure index.

With this configuration, the peripheral hemodynamic state can be estimated based on a combination of the pulse wave transmission time and the peripheral blood pressure index correlated with each other. The estimation accuracy of the peripheral hemodynamic state is thus improved.

In the above-described method, the estimating of the pulse wave transmission time may include continuously or intermittently measuring of the pulse wave transmission time of the user multiple times while the user is sleeping. The method may further include estimating the peripheral hemodynamic state and the quality of sleep of the user from a change in the pulse wave transmission time measured while the user is sleeping.

With this configuration, the quality of sleep can be estimated in addition to the peripheral hemodynamic state. This can increase the simplicity of estimating the quality of sleep.

The above-described method may further include determining the sleeping state of the user. With this arrangement, even if the pulse wave transmission time or the peripheral blood pressure index is changed when the user wakes up during sleep, for example, such a change does not influence the estimation of the quality of sleep. This improves the estimation accuracy of the quality of sleep.

In the above-described method, the estimating of the pulse wave transmission time may include continuously or intermittently measuring of the pulse wave transmission time of the user multiple times over a period of one day or longer. The method may further include estimating the peripheral hemodynamic state and a peripheral blood circulation disorder state of the user from a change in the pulse wave transmission time measured over the period of one day or longer.

With this configuration, the peripheral blood circulation disorder state can be estimated in addition to the peripheral hemodynamic state. As a result, the simplicity of estimating the peripheral blood circulation disorder state is increased.

The above-described method may further include determining whether the finger of the user is positioned at the height of the heart of the user. The estimating of the pulse wave transmission time may include estimating of the pulse wave transmission time when the finger is positioned at the height of the heart. The estimating of the peripheral hemodynamic state may include estimating of the peripheral hemodynamic state based on the pulse wave transmission time estimated at a time when the finger is positioned at the height of the heart. As a result, the peripheral hemodynamic state can be estimated with high accuracy by using only the pulse wave transmission time measured at a time when the finger is positioned at the height of the heart or by using a threshold for the pulse wave transmission time and that for the peripheral blood pressure index.

In the above-described method, the estimating of the pulse wave transmission time may include estimating of first and second pulse wave transmission times. The first pulse wave transmission time is a pulse wave transmission time when the finger of the user is positioned at a first height. The second pulse wave transmission time is a pulse wave transmission time when the finger of the user is positioned at a second height. The second height is different from the first height. The estimating of the peripheral hemodynamic state may include estimating of the peripheral hemodynamic state based on first and second peripheral hemodynamic states. The first peripheral hemodynamic state is a state estimated based on the first pulse wave transmission time. The second peripheral hemodynamic state is a state estimated based on the second pulse wave transmission time.

With this configuration, the peripheral hemodynamic state can be estimated by reducing the influence of variations in the pulse wave transmission time, which are caused by the difference in the height of the sensing device 20, on the estimation of the peripheral hemodynamic state. The estimation accuracy of the peripheral hemodynamic state is thus improved.

In the above-described method, the first height and the second height may be any one of combinations of: the height of the finger when the user is in a sitting posture and holds a hand of the user at a height of a chest of the user; the height of the finger when the user is in the sitting posture and holds the hand of the user at a height of a head of the user; and the height of the finger when the user is in a sitting posture and holds the hand of the user at a height of an abdomen of the user.

In the above-described method, the first height and the second height may be: the height of the finger when the user is in a supine posture on a flat surface and holds a hand of the user at a height of a chest of the user; and the height of the finger when the user is in the supine posture and holds the hand of the user at a height of the flat surface.

The above-described postures are those that the user can easily take, and that the user can repeat to make them highly reproducible. The peripheral hemodynamic state can thus be estimated with high reproducibility.

The above-described method may further include obtaining the amount of change of each of the first height and the second height. The estimating of the peripheral hemodynamic state may include estimating of the peripheral hemodynamic state based on the pulse wave transmission time and the amount of change of each of the first height and the second height. As a result, the influence of variations in the measurement position and variations in the estimation result of the pulse wave transmission time caused by the difference in the physical characteristics of users can be reduced. The estimation accuracy of the peripheral hemodynamic state is thus improved.

The above-described method may further include determining whether the user is in a resting state. The estimating of the peripheral hemodynamic state can include estimating of the peripheral hemodynamic state based on the pulse wave transmission time when the user is in the resting state.

With this configuration, the peripheral hemodynamic state can be estimated based on the pulse wave transmission time, which is less likely to change, thereby improving the estimation accuracy of the peripheral hemodynamic state.

The above-described embodiments are provided for facilitating the understanding of the exemplary aspects, but they are not intended to be exhaustive or to limit the invention to the precise forms disclosed. Modifications and/or improvements may be made without departing from the scope and spirit of the exemplary embodiment, and equivalents of the invention are also encompassed in the invention. That is, suitable design changes made to the embodiments by those skilled in the art are also encompassed in the invention within the scope and spirit of the invention. For example, the elements of the embodiments and the positions, conditions, shapes, and sizes thereof are not restricted to those described in the embodiments and may be changed in an appropriate manner. The embodiments are only examples, and the elements of the different embodiments may be partially replaced by or combined with each other, and configurations obtained by replacing or combining the elements of the embodiments are also encompassed in the invention within the scope and spirit of the present disclosure.

### REFERENCE SIGNS LIST

10 biological information measurement system, 20 sensing device, 21 biometric sensor, 211, 212 photoplethysmography sensor, 2111, 2121 light-emitting element, 213 light-receiving element, 22 control circuit, 23 communication module, 24 acceleration sensor, 25 housing, 30 computer, 31 communication module, 32 signal processing unit

1. A method for estimating a peripheral hemodynamic state of a user, the method comprising:
obtaining, by a first sensor that includes a first light-emitting device and at least one light-receiving device, a first photoplethysmographic signal of a capillary of a finger of a user;
obtaining, by a second sensor that includes a second light-emitting device and the at least one light-receiving device, a second photoplethysmographic signal of an arteriole of the finger of the user, the second light-emitting device being disposed farther away from the at least one light-receiving device than the first-light emitting device;
estimating, by at least one processor, a pulse wave transmission time based on the first and second photoplethysmographic signals; and
estimating, by the at least one processor, a peripheral hemodynamic state of the peripheral of the user based on the estimated pulse wave transmission time.

2. The method according to claim 1, further comprising estimating, by the at least one processor, a peripheral blood pressure index of the user based on the first and second photoplethysmographic signals.

3. The method according to claim 2, wherein the estimating of the peripheral hemodynamic state is based on the estimated pulse wave transmission time and the estimated peripheral blood pressure index.

4. The method according to claim 2, further comprising:
calculating, by the at least one processor, pulse wave feature values from the first and second photoplethysmographic signals; and
calculating, by the at least one processor, the peripheral blood pressure index of the user from the calculated feature values.

5. The method according to claim 1, further comprising continuously or intermittently measuring the pulse wave transmission time of the user a plurality of times while the user is sleeping to estimate the pulse wave transmission time.

6. The method according to claim 5, further comprising estimating, by the at least one processor, the peripheral hemodynamic state and a quality of sleep of the user from a change in the pulse wave transmission time measured while the user is sleeping.

7. The method according to claim 6, further comprising determining, by the at least one processor, a sleeping state of the user.

8. The method according to claim 1, further comprising continuously or intermittently measuring the pulse wave transmission time of the user a plurality of times over a period of one day or longer to estimate the pulse wave transmission time.

9. The method according to claim 8, further comprising estimating, by the at least one processor, the peripheral hemodynamic state and a peripheral blood circulation disorder state of the user based on a change in the pulse wave transmission time measured over the period of one day or longer.

10. The method according to claim 1, further comprising:
determining, by an acceleration sensor, whether the finger of the user is positioned at a height of a heart of the user;
estimating, by the at least one processor, the pulse wave transmission time when the finger is determined to be positioned at the height of the heart; and
estimating, by the at least one processor, the peripheral hemodynamic state based on the pulse wave transmission time estimated at a time when the finger is determined to be positioned at the height of the heart.

11. The method according to claim 1, wherein the estimating of the pulse wave transmission time includes estimating first and second pulse wave transmission times, the first pulse wave transmission time being a pulse wave transmission time when the finger of the user is positioned at a first height, the second pulse wave transmission time being a pulse wave transmission time when the finger of the user is positioned at a second height, and the second height being different from the first height.

12. The method according to claim 11, further comprising estimating the peripheral hemodynamic state based on first and second peripheral hemodynamic states, the first peripheral hemodynamic state being a state estimated based on the first pulse wave transmission time, and the second peripheral hemodynamic state being a state estimated based on the second pulse wave transmission time.

13. The method according to claim 12, wherein the first height and the second height are at least one of:
a height of the finger when the user is in a sitting posture and holds a hand at a height of a chest of the user,
a height of the finger when the user is in a sitting posture and holds the hand at a height of a head of the user, and
a height of the finger when the user is in a sitting posture and holds the hand at a height of an abdomen of the user.

14. The method according to claim 12, wherein the first height and the second height are a combination of:
a height of the finger when the user is in a supine posture on a flat surface and holds a hand at a height of a chest of the user; and
a height of the finger when the user is in the supine posture and holds the hand at a height of the flat surface.

15. The method according to claim 12, further comprising:
obtaining an amount of change of each of the first height and the second height; and
estimating, by the at least one processor, the peripheral hemodynamic state based on the pulse wave transmission time and the amount of change of each of the first height and the second height.

16. The method according to claim 1, further comprising:
determining, by the at least one processor, whether the user is in a resting state; and
estimating, by the at least one processor, the peripheral hemodynamic state based on the pulse wave transmission time when the user is in the resting state.

17. A method for estimating a peripheral hemodynamic state of a user, the method comprising:
obtaining, by a first photoplethysmographic sensor of a sensing device worn on a finger of a user, a first photoplethysmographic signal of a capillary of the finger of the user;
obtaining, by a second photoplethysmographic sensor of the sensing device, a second photoplethysmographic signal of an arteriole of the finger of the user;
estimating, by at least one processor, a pulse wave transmission time based on the first and second photoplethysmographic signals; and
estimating, by the at least one processor, a peripheral hemodynamic state of the peripheral of the user based on the estimated pulse wave transmission time.

18. The method according to claim 17, further comprising:
determining, by an acceleration sensor, whether the finger of the user is positioned at a height of a heart of the user;
estimating, by the at least one processor, the pulse wave transmission time when the finger is determined to be positioned at the height of the heart; and
estimating, by the at least one processor, the peripheral hemodynamic state based on the pulse wave transmission time estimated at a time when the finger is determined to be positioned at the height of the heart.

19. The method according to claim 17, wherein the estimating of the pulse wave transmission time includes estimating, by the at least one processor, first and second pulse wave transmission times, the first pulse wave transmission time being a pulse wave transmission time when the finger of the user is positioned at a first height, the second pulse wave transmission time being a pulse wave transmission time when the finger of the user is positioned at a second height, and the second height being different from the first height.

20. The method according to claim 12, further comprising estimating, by the at least one processor, the peripheral hemodynamic state based on first and second peripheral hemodynamic states, the first peripheral hemodynamic state being a state estimated based on the first pulse wave transmission time, and the second peripheral hemodynamic state being a state estimated based on the second pulse wave transmission time.
